(19) United States
(12) Patent Application Publication
(10) Pub. No.: US 2025/0261889 A1
(43) Pub. Date: Aug. 21, 2025

(54) STRESS ESTIMATION APPARATUS, METHOD, AND STORAGE MEDIUM

(71) Applicant: KABUSHIKI KAISHA TOSHIBA, Tokyo (JP)

(72) Inventor: Takashi Sudo, Fuchu Tokyo (JP)

(73) Assignee: KABUSHIKI KAISHA TOSHIBA, Tokyo (JP)

(21) Appl. No.: 19/043,977

(22) Filed: Feb. 3, 2025

(30) Foreign Application Priority Data
Feb. 16, 2024 (JP) 2024-021956

(51) Int. Cl. A61B 5/16; A61B 5/00; A61B 5/0295; A61B 5/28

(52) U.S. Cl. CPC A61B 5/165; A61B 5/0082; A61B 5/0295; A61B 5/28; A61B 5/6823; A61B 5/6824; A61B 5/6897; A61B 5/7264; A61B 5/746; A61B 2560/0462

(57) ABSTRACT

According to one embodiment, a stress estimation apparatus includes a processing circuit and an acquirer. The processing circuit detects at least one of a work start time and a work end time for a user performing work during working hours. The processing circuit calculates, based on the detected result, a short-term stress value from a biometric waveform of the user acquired by the acquirer. The processing circuit calculates a baseline based on at least the working-hours short-term stress value. The processing circuit calculates a stress fluctuation range of the user by removing the baseline from the short-term stress value. The processing circuit estimates, based on the stress fluctuation range, a magnitude of long-term stress of the user.

### CROSS-REFERENCE TO RELATED APPLICATION

This application is based upon and claims the benefit of priority from Japanese Patent Application No. 2024-021956, filed Feb. 16, 2024, the entire contents of which are incorporated herein by reference.

### FIELD

Embodiments described herein relate generally to a stress estimation apparatus, a method, and a storage medium.

### BACKGROUND

In recent years, a method for estimating the magnitude of the long-term stress of a user has been known. According to this type of method, for example, an autonomic nerve index indicating a variation in heartbeat (or pulse) is calculated based on a heartbeat interval (or pulse interval) of one minute acquired from the user, and the magnitude of long-term stress of the user is estimated from a short-term stress value determined from the autonomic nerve index. Note that, in this method, because individual differences in the autonomic nerve index are large, a baseline is determined for each individual, and it is assumed that an increase from the baseline corresponds to the magnitude of long-term stress. Specifically, the user is allowed to stand still and rest before starting work, a first short-term stress value before the start of work is set as the baseline, and the stress corresponding to the increase from the baseline is measured as the response to the work load. That is, in this method, the first short-term stress value before the start of work and a second short-term stress value during work are calculated. Thereafter, the magnitude of the long-term stress is estimated according to the increase from the baseline by subtracting the first short-term stress value (baseline) from the second short-term stress value after the end of work.

However, according to a study by the present inventors, it is difficult to apply a method like the method described above to an office worker among various users. For example, office workers are often unable to remain calm and restful at the beginning of the workday, before the start of work, due to delays in work progress or transportation delays. In such cases, there is an increase in heart rate (or pulse) before the start of work, the first short-term stress value (baseline) rises, and thus the estimated long-term stress decreases. Note that another office worker may not be able to be calm and restful at the work end time, i.e. after the end of work, due to delays in work progress or circumstances that preclude overtime work. In such cases, there is an increase in heart rate (or pulse) after the end of work, the second short-term stress value rises, and thus the estimated long-term stress increases. That is, for a user such as an office worker, stress is likely to arise at the start or end of work, and thus, the estimated long-term stress increases or decreases, and it is difficult to apply the above-described method. It is therefore desirable that the magnitude of long-term stress be appropriately estimated for a user who is likely to experience stress at the start or end of work.

### BRIEF DESCRIPTION OF DRAWINGS

FIG. 1 is a block diagram showing an example of a configuration of a stress estimation apparatus according to a first embodiment.

FIG. 2 is a flowchart to illustrate operations according to the first embodiment.

FIG. 3 is a schematic diagram to illustrate an operation according to the first embodiment.

FIG. 4 is a block diagram showing an example of a configuration of a stress estimation apparatus according to a second embodiment.

FIG. 5 is a flowchart to illustrate operations according to the second embodiment.

FIG. 6 is a block diagram showing an example of a configuration of a stress estimation apparatus according to a third embodiment.

FIG. 7 is a schematic diagram to illustrate a regression model dictionary according to the third embodiment.

FIG. 8 is a flowchart to illustrate operations according to the third embodiment.

FIG. 9 is a schematic diagram to illustrate a modification according to the third embodiment.

FIG. 10 is a schematic diagram to illustrate a modification according to the third embodiment.

FIG. 11 is a block diagram showing an example of a configuration of a stress estimation apparatus according to another modification of the third embodiment.

FIG. 12 is a flowchart to illustrate operations according to another modification of the third embodiment.

FIG. 13 is a schematic diagram to illustrate operations according to a fourth embodiment.

FIG. 14 is a schematic diagram to illustrate a modification according to the fourth embodiment.

FIG. 15 is a block diagram showing an example of a hardware configuration of a stress estimation apparatus according to a fifth embodiment.

FIG. 16 is a block diagram showing an example of a hardware configuration of a stress estimation system including the stress estimation apparatus according to a modification of the fifth embodiment.

### DETAILED DESCRIPTION

In general, according to one embodiment, a stress estimation apparatus includes a processing circuit and an acquirer. The processing circuit is configured to detect at least one of a work start time and a work end time for a user performing work during working hours between the work start time and the work end time. The processing circuit is configured to calculate, based on the detected result, a short-term stress value from a biometric waveform of the user acquired by the acquirer. The processing circuit is configured to calculate a baseline based on at least the working-hours short-term stress value. The processing circuit is configured to calculate a stress fluctuation range of the user by removing the baseline from the calculated short-term stress value. The processing circuit is configured to estimate, based on the stress fluctuation range, a magnitude of long-term stress of the user.

Hereinafter, a stress estimation apparatus, a method, and a storage medium according to each embodiment will be described with reference to the drawings. In each of the following embodiments, parts denoted by the same reference signs perform similar operations, and redundant descriptions will be omitted as appropriate.

### First Embodiment

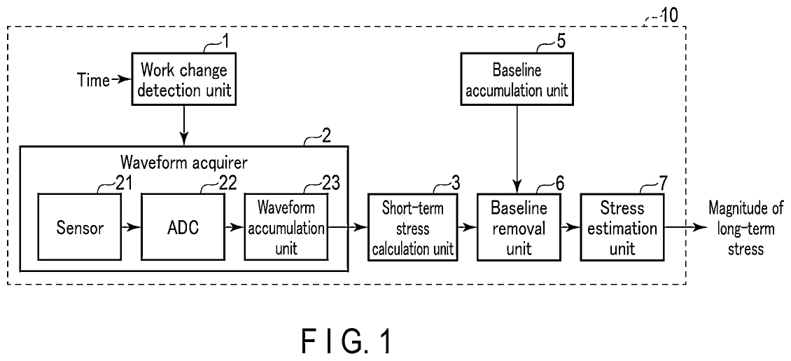

FIG. 1 is a block diagram showing an example of a configuration of a stress estimation apparatus according to a first embodiment. A stress estimation apparatus 10 includes a work change detection unit 1, a waveform acquirer 2, a short-term stress calculation unit 3, a baseline accumulation unit 5, a baseline removal unit 6, and a stress estimation unit 7.

Here, the work change detection unit 1 detects at least one of a work start time and a work end time for a user performing work during working hours between the work start time and work end time. For example, the work change detection unit 1 may detect the start of work from a startup operation of the user, or may detect the end of work from an end operation by the user. Note that the subject of the startup operation and the end operation may be a user PC (personal computer) or a login application on the user PC. That is, the work change detection unit 1 may be disposed in a user PC which is an office device used for the work of the user. In this case, the work change detection unit 1 may detect at least one of the work start time and the work end time in response to the operations of the user on the office device used for the work. Here, the office device signifies, for example, a user-dedicated device in the office, such as a user PC. In addition, the office device does not mean a shared device in the office, such as a printer, a copier, and a shredder, for example. Further, in a case where the work start time is to be detected, the work change detection unit 1 may detect the work start time in the morning and the work start time in the afternoon separately according to the startup operation and the time. For example, the start time in the afternoon is detected in a case where work is started twice a day, such as when work is started in the afternoon (after lunch break) after a morning break or attendance at work is in the morning and at night. Furthermore, the working hours are not detected because same are obtained as a fixed time period after the start of work is detected. The work change detection unit 1 is an example of a detection unit.

The waveform acquirer 2 acquires a biometric waveform of the user based on the detected result. For example, in a case where the work change detection unit 1 detects the start of work, the waveform acquirer 2 acquires a biometric waveform at the detected work start time. In addition, for example, in a case where the work change detection unit 1 detects the end of work, the waveform acquirer 2 acquires a detected biometric waveform at the end of work. Specifically, for example, the waveform acquirer 2 includes a sensor 21, an ADC 22, and a waveform accumulation unit 23. ADC is an abbreviation for analog-digital converter. The sensor 21 detects biometric data from the user. As the biometric data, for example, at least one of a pulse waveform and an electrocardiographic waveform can be used as appropriate. The ADC 22 performs A/D conversion on the analog data output from the sensor 21 and outputs the digital data. For example, the pulse waveform or the electrocardiographic waveform is converted into digital data at a sampling interval of 1 [ms](sampling frequency 1 k [Hz]). The waveform accumulation unit 23 accumulates the digital data chronologically. The accumulated digital data corresponds to a biometric waveform representing biometric data chronologically. In addition, as the waveform acquirer 2, a configuration for acquiring a biometric waveform from a user by means of biometric sensors can be used as appropriate. The biometric sensors may include, for example, at least one of an electrocardiographic sensor, a photoelectric pulse wave sensor, a chestband sensor, a wristband sensor, a mouse-mounted sensor, and an optical camera. As the mouse-mounted sensor, for example, a photoelectric pulse wave sensor mounted in a position on the mouse of the user PC where the fingertips of the user hit can be used as appropriate. In this case, only biometric waveforms that can be stably collected by the fingertips of the user continuing to make stable contact may be accumulated. The optical camera is mounted on, for example, a smartphone, and is used in a case where moving image data of the fingertips or face of the user is photographed, and a biometric waveform of a pulse waveform corresponding to a change in color of the fingertips or a change in complexion is acquired from a change in a green component of the moving image data. The chestband, the wristband, and the smartphone of a biometric sensor are examples of devices that can be carried by the user. That is, the waveform acquirer 2 may be disposed in a device that can be carried by the user. Note that the present invention is not limited to such placement, rather, the waveform acquirer 2 may, for example, also be disposed in an office device used for the work of the user as in a case where an optical camera is mounted on a user PC. The waveform acquirer 2 is an example of an acquirer.

The short-term stress calculation unit 3 calculates a short-term stress value from a biometric waveform. The short-term stress calculation unit 3 calculates various short-term stress values by obtaining, from the electrocardiographic waveform, an RR interval (RRI, heartbeat interval), which is an interval between heartbeats, or by obtaining, from the pulse waveform, a pulse wave peak interval (PPI, Pulse Interval) which is the peak interval thereof and analyzing fluctuations in the RRI or the PPI. For example, the short-term stress calculation unit 3 calculates a short-term stress value at the work start time from a biometric waveform at the work start time. In addition, for example, the short-term stress calculation unit 3 calculates a work-end-time short-term stress value from a biometric waveform at the work end time. Furthermore, as the short-term stress value, an autonomic nerve index related to variations in a heartbeat interval or a pulse interval can be used. For example, as the short-term stress value, RR_mean, RR_std, CV, NN50, pNN50, NN40, pNN40, NN30, pNN30, RMSSD, LF, HF, and LF/HF can be used as appropriate. Here, RR_mean is the heartbeat interval average and represents the average of a fixed segment of heartbeat intervals. RR_std is the standard deviation of the heartbeat intervals and represents the standard deviation of a fixed segment of heartbeat intervals. CV is the coefficient of variation of the heartbeat interval, and represents a coefficient of variation (standard deviation/average) of a fixed segment of heartbeat intervals. The NN50 represents the number of adjacent heartbeat intervals having a difference of 50 [ms] or more. pNN50 represents the proportion of NN50. The NN40 represents the number of adjacent heartbeat intervals having a difference of 40 [ms] or more. pNN40 represents the proportion of NN40. The NN30 represents the number of adjacent heartbeat intervals having a difference of 30 [ms] or more. pNN30 represents the proportion of NN30. RMSSD is an abbreviation for Root Mean Square of Successive Differences and represents the square root of the mean square of the difference between consecutive adjacent heartbeat intervals. LF is an abbreviation for low frequency, and represents a low frequency component (0.04 to 0.15 [Hz]) of the heartbeat interval. HF is an abbreviation for High Frequency and represents a high-frequency component (0.15 to 0.40 [Hz]) of the heartbeat interval. LF/HF is the ratio of LF to HF and represents the balance between the degree of the sympathetic tone (LF) and the degree of the parasympathetic tone (HF). That is, as the short-term stress value, various autonomic nerve indexes in a frequency domain or a time domain can be used. Here, the fixed segment signifies the time width to be analyzed, and is, for example, 60 [s]. In this case, the short-term stress value is calculated at intervals of at least 60 [s]. In addition, although the method of calculating various short-term stress values using the heartbeat interval as an input has been described here, calculation is similarly performed even in a case where the pulse interval is used as an input. Furthermore, various known methods can be applied to the calculation of the short-term stress value. The short-term stress calculation unit 3 is an example of a calculation unit.

The baseline accumulation unit 5 calculates a baseline based on at least the working-hours short-term stress values and accumulates the baseline in the memory. Here, the working hours start, for example, after a fixed time period from the start of work. Furthermore, regardless of the nature of the work during working hours, the working hours can be switched from the start of work by a predetermined time frame after the time the work is started. In addition, the baseline may be the smallest value among the work-start-time short-term stress values, the working-hours short-term stress values, and the work-end-time short-term stress values. Further, the baseline may be the smallest value among an average value of the work-start-time short-term stress values, an average value of the working-hours short-term stress values, and an average value of the work-end-time short-term stress values. In any case, the working-hours short-term stress values are taken into account in calculating the baseline. The baseline accumulation unit 5 is an example of a baseline calculation unit.

The baseline removal unit 6 calculates the stress fluctuation range of the user by removing the baseline from the calculated short-term stress values. For example, the baseline removal unit 6 calculates the work-start-time stress fluctuation range by removing the baseline from the work-start-time short-term stress values. Further, the stress estimation apparatus 10 typically performs stress measurement at the start of work when short-term stress is likely to appear, and uses a long-time average or the smallest value among the short-term stress values measured during working hours as a baseline for the short-term stress values for each individual in order to exclude idiosyncrasies. The long-time average and the smallest value of the working-hours short-term stress values reflect a period of being calm and at rest during working hours. The baseline removal unit 6 calculates, as a stress fluctuation range at the work start time, a difference (average fluctuation range at the work start time) obtained by removing the baseline from the work-start-time short-term stress values. In addition, for example, the baseline removal unit 6 calculates a work-end-time stress fluctuation range by removing the baseline from the work-end-time short-term stress values. The work-end-time stress fluctuation range signifies the average fluctuation range at the work end time, as described above. Note that the work-start-time stress fluctuation range and the work-end-time stress fluctuation range are each used to estimate stress values on a daily basis. The baseline removal unit 6 is an example of a removal unit.

The stress estimation unit 7 estimates the magnitude of the long-term stress of the user based on the stress fluctuation range. The estimated magnitude of the long-term stress is output on a daily basis by taking, as an input, the stress fluctuation width on a daily basis. As the magnitude of long-term stress, as in the case of a subjective questionnaire, for example, a subjective evaluation value obtained by scoring answers of various users to a plurality of items of questions can be used as appropriate. For the subjective evaluation value, for example, values of subjective evaluation such as 21 items of BDI-II asking about the depression scale of the most recent 2 to 3 days, 20 items of “Depressive Self-Rating Scale” (SDS) asking about the current depression scale, 40 items of STAI “state characteristic anxiety test” asking about the current state and usual feeling, 57 items of occupational stress simple questionnaire asking about the state in the most recent single month, 65 items of POMS2 asking about the feeling in the most recent single week, and 10 items of PSS-10 asking about the feeling and behavior in the most recent single month can be used as appropriate. BDI-II is an abbreviation for Beck Depression Inventory-Second Edition. SDS is an abbreviation for Self-rating Depression Scale. STAI is an abbreviation for State-Trait Anxiety Inventory. POMS2 is an abbreviation for Profile of Mood States 2nd Edition. PSS is an abbreviation for Perceived Stress Scale. For example, the stress estimation unit 7 may have a function for calculating the subjective evaluation value from the stress fluctuation range based on an objective biometric waveform, and may use the function to estimate the magnitude of the long-term stress, which is the subjective evaluation value, from the stress fluctuation range. As the function, for example, a regression model (regression equation) representing the relationship between the stress fluctuation range, which is an explanatory variable, and the magnitude of the long-term stress, which is a response variable, can be used as appropriate. This regression model is created in advance from the relationship between the stress fluctuation range and the magnitude of long-term stress in an unspecified large number of people. In addition, the regression model is a model that represents the relationship between an objective value (stress fluctuation range) such as an autonomic nerve index and a subjective value (magnitude of long-term stress) such as the score of a subjective questionnaire. The regression model may be a single regression model or a multiple regression model, or may be a linear regression model or a nonlinear regression model. Furthermore, the model may be a model using machine learning such as Lasso regression, Ridge regression, ElasticNet regression, LightGBM, or random forest regression. The regression model may be referred to as a regression equation. The stress estimation unit 7 is an example of an estimation unit.

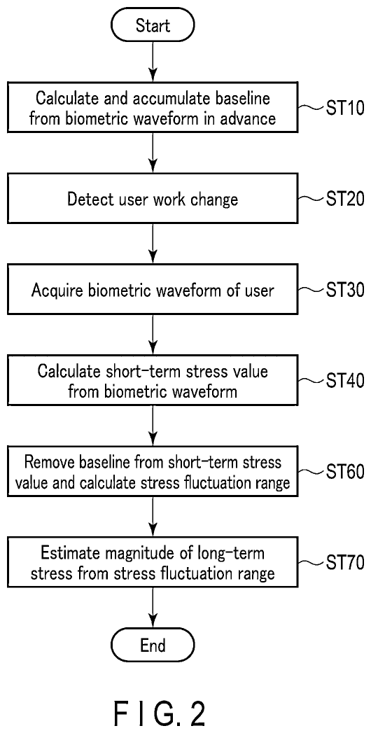

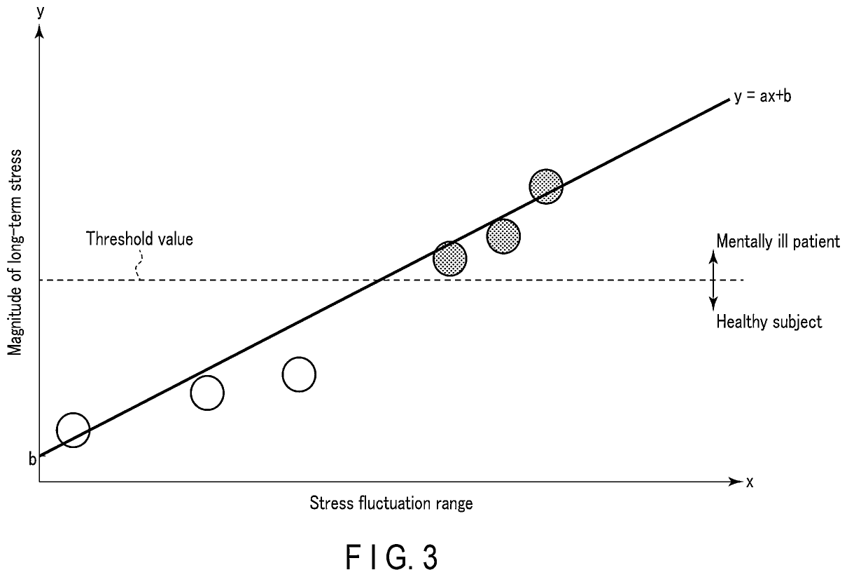

Next, the operation of the stress estimation apparatus configured as detailed above will be described with reference to the flowchart of FIG. 2 and the schematic diagram of FIG. 3.

### (Step ST10)

The baseline accumulation unit 5 calculates and accumulates, in advance, a baseline for the short-term stress values from the biometric waveform of the user. Specifically, the baseline accumulation unit 5 calculates a baseline based on at least the working-hours short-term stress values among the short-term stress values calculated from the biometric waveforms at the work start time, during working hours, and at the work end time, and accumulates the baseline in the memory. In this example, it is assumed that the short-term stress value is RR_mean (heartbeat interval average). In addition, it is assumed that the baseline is the smallest value among RR_mean at the work start time, RR_mean during working hours, and RR_mean at the work end time.

### (Step ST20)

The work change detection unit 1 detects at least one of the work start time and the work end time for a user performing work during working hours between the work start time and the work end time. For example, the work change detection unit 1 detects the start of work in response to a login application startup operation by the user.

### (Step ST30)

The waveform acquirer 2 acquires the biometric waveform of the user based on the result detected in step ST20. For example, the waveform acquirer 2 acquires the detected electrocardiogram waveform at the start of work as a biometric waveform.

### (Step ST40)

The short-term stress calculation unit 3 calculates a short-term stress value from the biometric waveform acquired in step ST30. For example, the short-term stress calculation unit 3 calculates a short-term stress value at the work start time from a biometric waveform at the work start time. Specifically, for example, the short-term stress calculation unit 3 calculates RR_mean (the heartbeat interval average) as the short-term stress value from an electrocardiogram waveform at the start of work.

### (Step ST60)

The baseline removal unit 6 calculates the stress fluctuation range of the user by removing the baseline from the short-term stress value calculated in step ST40. In this example, the stress fluctuation range represents a fluctuation range (heart rate fluctuation) of RR_mean at the start of work.

### (Step ST70)

The stress estimation unit 7 estimates the magnitude of the long-term stress of the user based on the stress fluctuation range calculated in step ST60. For example, as illustrated in FIG. 3, the stress estimation unit 7 performs estimation by using a regression model y=ax+b representing the relationship between the stress fluctuation width and the long-term stress when the stress fluctuation width is x and the magnitude of the long-term stress is y. Note that, to simplify the description, a simple first-order linear regression is used, and in this regression model, a represents a regression coefficient, and b represents a y-intercept. In addition, the stress estimation unit 7 may estimate a user for whom the estimated magnitude of long-term stress exceeds a threshold value as a person suffering from a mental disorder (a high-stress person), and may estimate a user for whom the magnitude is equal to or less than the threshold value as a healthy person (low-stress person). In any case, the stress estimation unit 7 outputs an estimation result including the magnitude of long-term stress to a display or the like. Thereafter, the stress estimation apparatus 10 ends the processing.

As described above, according to the first embodiment, the work change detection unit 1 detects at least one of the work start time and the work end time for a user performing work during working hours between the work start time and the work end time. The waveform acquirer 2 acquires a biometric waveform of the user based on the detected result. The short-term stress calculation unit 3 calculates a short-term stress value from a biometric waveform. The baseline accumulation unit 5 calculates a baseline based on at least the working-hours short-term stress values. The baseline removal unit 6 calculates the stress fluctuation range of the user by removing the baseline from the calculated short-term stress values. The stress estimation unit 7 estimates the magnitude of the long-term stress of the user based on the stress fluctuation range. As described above, with a configuration in which the baseline, which is based on at least the working-hours short-term stress value, is removed from the short-term stress value at the time of starting and/or ending work, the magnitude of the long-term stress can be appropriately estimated for a user who is likely to experience stress at the time of starting or ending work. In addition, in the first embodiment, unlike conventional cases, the magnitude of the long-term stress can be appropriately estimated by means of a configuration in which the short-term stress values at the start and end of work, which are likely to appear as stress, are not used as the baseline, and thus an increase or decrease in the estimated long-term stress is suppressed. Furthermore, with a configuration in which the baseline for the user is removed from the short-term stress values of the user, an improvement in the accuracy of the stress fluctuation range can be expected.

Furthermore, according to the first embodiment, the work change detection unit 1 detects the start of work. The waveform acquirer 2 acquires the detected biometric waveform at the work start time. The short-term stress calculation unit 3 calculates a short-term stress value at the work start time from a biometric waveform at the work start time. The baseline removal unit 6 calculates a work-start-time stress fluctuation range by removing the baseline from the work-start-time short-term stress values. Therefore, similarly to the above-described advantageous effect, the magnitude of the long-term stress of the user at the start of work can be appropriately estimated.

In addition, according to the first embodiment, the work change detection unit 1 may detect the work end time. In a case where the work change detection unit 1 detects the work end time, the waveform acquirer 2 acquires a biometric waveform at the detected work end time. The short-term stress calculation unit 3 calculates a work-end-time short-term stress value from the work end time biometric waveform. The baseline removal unit 6 calculates a work-end-time stress fluctuation range by removing the baseline from the work-end-time short-term stress values. Therefore, in this case, similarly to the above-described advantageous effects, the magnitude of the long-term stress of the user at the end of work can be appropriately estimated.

According to the first embodiment, the baseline is the smallest value among the work-start-time short-term stress values, the working-hours short-term stress values, and the short-term stress values at the work end time. Therefore, in addition to the above-described advantageous effects, it is possible to take, as the baseline, the short-term stress value when the user is most calm and resting during all of the periods of the work start time, during working hours, and the work end time. For this reason, it is possible to calculate an appropriate baseline even in a case where it is difficult for the user to be calm and rest.

In addition, according to the first embodiment, the baseline may be the smallest value among the average value of the work-start-time short-term stress values, the average value of the working-hours short-term stress values, and the average value of the work-end-time short-term stress values. In this case, in addition to the above-described advantageous effects, when one short-term stress value becomes the smallest value (an anomalous value) due to some anomaly, the smallest value of the average values obtained including the anomalous value is set as the baseline, and thus the effect of the anomalous value can be mitigated.

In addition, according to the first embodiment, the work change detection unit 1 detects the start of work from a startup operation by the user. Further, the work change detection unit 1 may detect the work end time from an end operation by the user. Therefore, in addition to the above-described advantageous effects, it is possible to appropriately detect the work start time and the work end time. In addition, in a case where the start of work is simply detected based on time, there is a disadvantage that the start of work cannot be detected when the user goes to work in a time frame that does not include the time used for detection, such as when the user is on morning leave or is late. In contrast, according to the first embodiment, because the start time is detected from a startup operation by the user, the work start time can be appropriately detected. The same applies to detection at the end of work.

Furthermore, according to the first embodiment, in a case where the work start time is to be detected, the work change detection unit 1 may detect the work start time in the morning and the work start time in the afternoon separately according to the startup operation and the time. In this case, the above-described advantageous effects can also be obtained for a work schedule where work is started in the afternoon exists, such as at a workplace where the start of work is in the afternoon after morning leave or at a workplace where work is started twice per day as in the case of morning and evening work.

### Second Embodiment

Next, a stress estimation apparatus according to a second embodiment will be described.

The second embodiment is a modification of the first embodiment and represents a specific example in a case where the processing of the short-term stress values is switched between the work start time and during working hours.

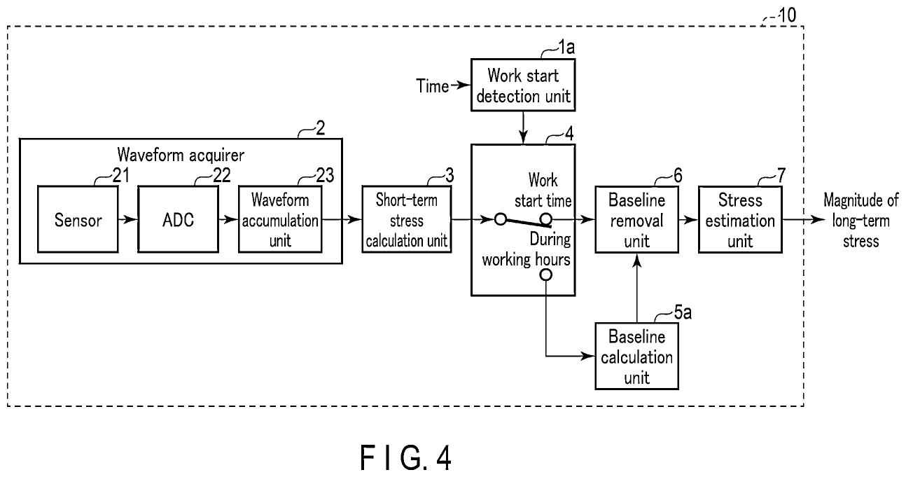

FIG. 4 is a block diagram showing an example of a configuration of a stress estimation apparatus according to the second embodiment. As compared with the configuration illustrated in FIG. 1, the stress estimation apparatus 10 includes a work start detection unit 1a and a baseline calculation unit 5a instead of the work change detection unit 1 and the baseline accumulation unit 5, and further includes a work timing determination unit 4.

Here, the work start detection unit 1a is a specific example of the work change detection unit 1, and detects the start of work of a user performing work during working hours between the work start time and the work end time. Note that the method for detecting the work start time by the work start detection unit 1a is similar to the method by the work change detection unit 1.

The work timing determination unit 4 sends the short-term stress values calculated by the short-term stress calculation unit to at least one of the baseline calculation unit 5a and the baseline removal unit 6 based on the result detected by the work start detection unit 1a. For example, the work timing determination unit 4 sends the work-start-time short-term stress values to the baseline removal unit 6 based on the result of detecting the work start time. Furthermore, the work timing determination unit 4 determines the working hours start timing according to whether or not a certain period of time has elapsed since the timing at which the start of work is detected, and thus sends a working-hours short-term stress value to the baseline calculation unit 5a. The work timing determination unit 4 is an example of a sending unit.

The baseline calculation unit 5a is a specific example of the baseline accumulation unit 5 and calculates a baseline based on the working-hours short-term stress values thus sent, and accumulates the baseline in the memory.

The baseline removal unit 6 calculates the user stress fluctuation range by removing the baseline from the sent short-term stress values.

Other configurations are similar to that of the first embodiment.

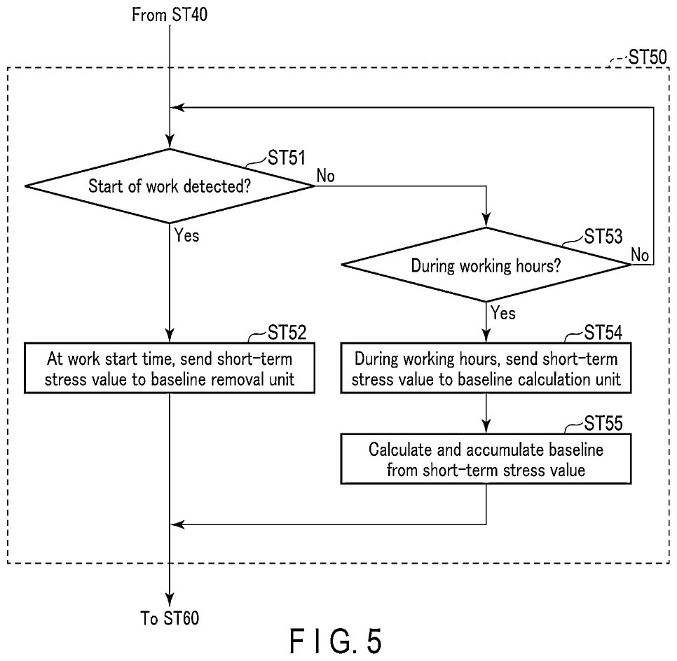

Next, the operation of the stress estimation apparatus configured as described above will be described with reference to the flowchart of FIG. 5. Note that, in FIG. 5, step ST50, which is circled by the broken line, is added between step ST40 and step ST60 shown in FIG. 2. In the present embodiment, step ST10 of accumulating the baseline in advance, as shown in FIG. 2, is omitted.

First, step ST20 is executed in the same manner as described above, and the work start detection unit 1a detects the start of work of a user performing work during working hours between the start work time and the work end time.

Similarly, steps ST30 to ST40 are executed, the biometric waveform of the user is acquired, and the short-term stress value is calculated from the biometric waveform. Thereafter, step ST50 is performed. Step ST50 includes steps ST51 to ST55.

### (Step ST50)

The work timing determination unit 4 determines whether the work start time has been detected by the work start detection unit 1a (step ST51). In a case where the start work time is detected as a result of this determination, the work timing determination unit 4 sends the short-term stress value at the start work time to the baseline removal unit 6 (step ST52) and advances to step ST60.

On the other hand, in a case where the determination result of step ST51 is negative, the work timing determination unit 4 determines the working hours start timing according to whether a fixed time period has elapsed since the timing at which the start of work is detected (step ST53). In the case of a negative determination result, the processing returns to step ST51.

In a case where the result of the determination in step ST53 is the working hours start timing, the work timing determination unit 4 sends a working-hours short-term stress value to the baseline calculation unit 5a (step ST54).

After step ST54, the baseline calculation unit 5a calculates a baseline based on the sent working-hours short-term stress value, and accumulates the baseline in the memory (step ST55). Upon completion of step ST52 or ST55, step ST50 ends.

Hereinafter, the processing in and after step ST60 is executed in the same manner as described above. In the case of the second embodiment, the baseline removal unit 6 calculates the stress fluctuation range of the user at the start of work by removing the baseline during working hours from the work-start-time short-term stress values (step ST60).

The stress estimation unit 7 estimates the magnitude of the long-term stress of the user based on the work-start-time stress fluctuation range (step ST70), and outputs the estimation result to a display or the like.

As described above, according to the second embodiment, the work timing determination unit 4 sends the short-term stress values to at least one of the baseline calculation unit 5a and the baseline removal unit 6 based on the result detected by the work start detection unit 1a. Therefore, in addition to the above-described advantageous effects, with a configuration in which the latest short-term stress values are used for the baseline calculation or baseline removal, the magnitude of the long-term stress of the user can be estimated based on the latest baseline.

### Third Embodiment

Next, a stress estimation apparatus according to a third embodiment will be described.

The third embodiment is a modification of the second embodiment, and represents a specific example in a case where a regression model or a threshold value determination is used at the time of stress estimation.

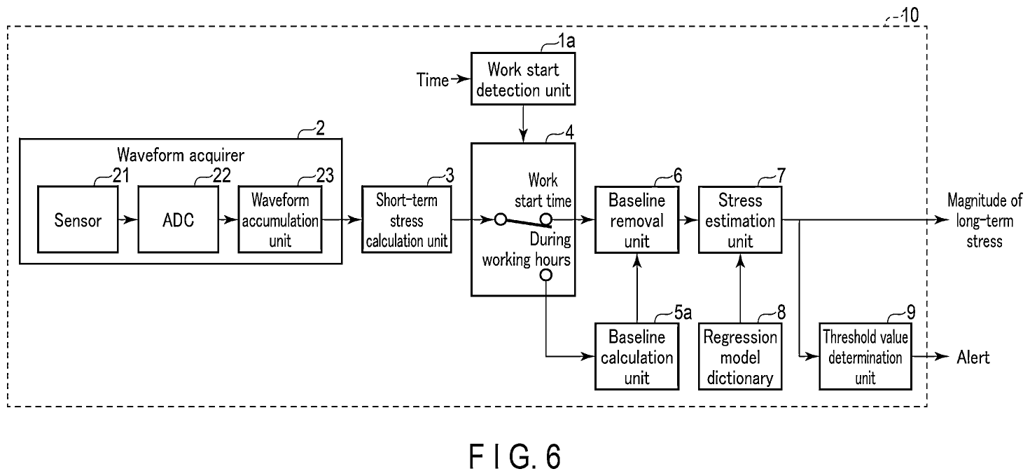

FIG. 6 is a block diagram showing an example of a configuration of a stress estimation apparatus according to the third embodiment. The stress estimation apparatus 10 further includes a regression model dictionary 8 and a threshold value determination unit 9 as compared with the configuration illustrated in FIG. 4.

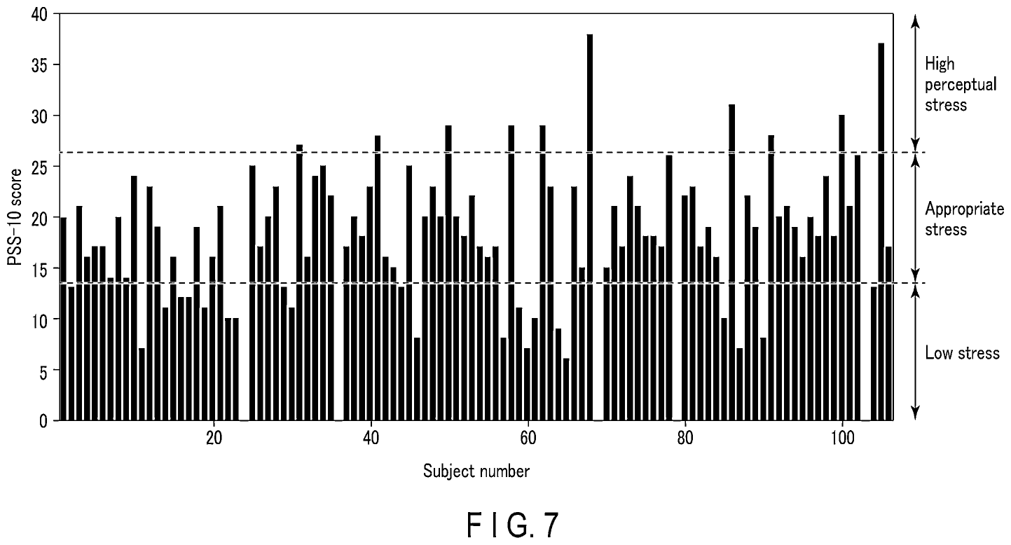

Here, as illustrated in FIG. 3, the regression model dictionary 8 stores a regression model representing the relationship between the stress fluctuation range and the magnitude of long-term stress in advance. Specifically, the regression model is created from the result of examining the relationship between the stress fluctuation range and the magnitude of long-term stress in advance for an unspecified multiplicity of subjects. For example, as illustrated in FIG. 7, the magnitude of long-term stress is obtained as a subjective evaluation value (e.g. a PSS-10 score) for an unspecified multiplicity of subjects. In FIG. 7, the vertical axis represents the PSS-10 score, and the horizontal axis represents the subject number. In the range of 0 point to 40 points, a PSS-10 score less than 14 points is defined as low stress, a score of 14 points or more and less than 27 points is defined as moderate stress, and a score of 27 points or more is defined as high perceptual stress. Note that the subjective evaluation value is not limited to the PSS-10 score, and scores such as BDI-II, SDS, STAI, the Brief Survey of Occupational Stress, and POMS2 can be used as appropriate, in the same manner as described above. The stress fluctuation range is the same type of objective evaluation value as the short-term stress value described above. As described above, examples of types of stress fluctuation and short-term stress that can be used, as appropriate, are RR_mean, RR_std, CV, NN50, pNN50, NN40, pNN40, NN30, pNN30, RMSSD, LF, HF, and LF/HF. In addition, for example, the regression model dictionary 8 stores a regression model for each type of stress fluctuation width in advance. That is, the regression model dictionary 8 stores the regression model in association with the type of the stress fluctuation range, for example, by storing the first regression model in association with the pulse wave peak interval and storing the second regression model in association with the RR_mean. The regression model dictionary 8 is an example of a storage unit.

Accordingly, the stress estimation unit 7 estimates the magnitude of the long-term stress of the user based on the regression model in the regression model dictionary 8. In addition, the stress estimation unit 7 estimates the magnitude of the long-term stress of the user based on the regression model corresponding to the type of the calculated stress fluctuation range.

The threshold value determination unit 9 outputs an alert in a case where the magnitude of the long-term stress estimated by the stress estimation unit 7 exceeds a threshold value. For example, in FIG. 3, in a case where the magnitude of long-term stress is a score of PSS-10, 27 points can be used as the threshold value. However, the threshold value is not limited thereto, and another score (for example, 23 points for a case of detecting the risk of early long-term stress) which is appropriate as the threshold value may be used even in the case of the PSS-10 score. In addition, for example, the threshold value determination unit 9 compares the estimated magnitude of the long-term stress with the threshold value, and outputs an alert according to a result of the comparison. The threshold value determination unit 9 is an example of an output unit.

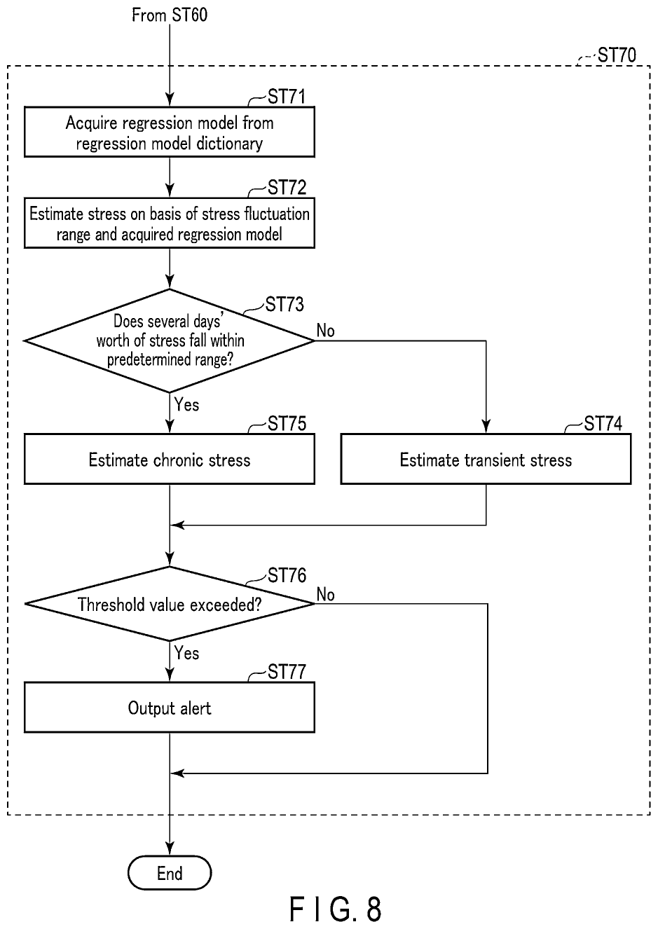

Next, the operation of the stress estimation apparatus configured as described above will be described with reference to the flowchart of FIG. 8. Note that, in FIG. 8, step ST70 surrounded by a broken line is executed as a specific example of step ST70 of the second embodiment.

Now, it is assumed that steps ST20 to ST60 are executed in the same manner as described above, and that the stress fluctuation range of the user at the start of work is calculated in step ST60.

### (Step ST70)

In step ST70, the stress estimation unit 7 estimates the magnitude of the long-term stress of the user based on the work-start-time stress fluctuation range. Such a step ST70 includes steps ST71 to ST77.

The stress estimation unit 7 acquires a regression model from the regression model dictionary 8 based on the type of stress fluctuation range (step ST71). In addition, the stress estimation unit 7 estimates the magnitude of the long-term stress based on the calculated stress fluctuation range and the acquired regression model (step ST72), outputs the estimation result to a display or the like, and stores the magnitude of the long-term stress in memory in association with the date.

After step ST72, the stress estimation unit 7 determines whether or not the magnitude of several days' worth of long-term stress falls within a predetermined range (step ST73), and if not, estimates the estimation result of step ST72 as transient stress (step ST74). If the result of the determination in step ST73 is within the predetermined range, the estimation result in step ST72 is estimated as chronic stress (step ST75). In addition, the stress estimation unit 7 outputs the estimation result of step ST74 or ST75 to a display or the like, and advances to step ST76.

In step ST76, the threshold value determination unit 9 determines whether or not the magnitude of the long-term stress estimated in step ST72 has exceeded the threshold value, and if not, terminates the processing. In addition, in a case where the threshold value is exceeded as a result of the determination in step ST76, the threshold value determination unit 9 outputs an alert to a display or the like (step ST77), and ends the processing. Note that steps ST76 to ST77 may be executed between steps ST72 and ST73.

As described above, according to the third embodiment, the regression model dictionary 8 stores a regression model representing the relationship between the stress fluctuation range and the magnitude of long-term stress in advance. Therefore, in addition to the above-described advantageous effects, the magnitude of long-term stress can be easily estimated by using a pre-stored regression model.

In addition, according to the third embodiment, the regression model dictionary 8 stores a regression model for each type of stress fluctuation width in advance. The stress estimation unit 7 estimates the magnitude of the long-term stress of the user based on the regression model corresponding to the type of the calculated stress fluctuation range. Therefore, in addition to the above-described advantageous effects, because the magnitude of long-term stress can be estimated using the regression model corresponding to the type from various types of stress fluctuation ranges, versatility can be improved.

In addition, according to the third embodiment, the threshold value determination unit 9 outputs an alert in a case where the estimated magnitude of the long-term stress exceeds the threshold value. Therefore, in addition to the above-described advantageous effects, it is possible, by means of an alert, to prompt a user suffering from significant long-term stress to take action such as rest.

Further, according to the third embodiment, in a case where the magnitude of several days' worth of long-term stress falls within a predetermined range, chronic stress can be detected instead of transient stress. In addition, the stress estimation apparatus 10 is capable of accurately estimating daily stress values, and therefore it is possible to observe daily changes, and to detect, for example, stress which is chronic (chronic stress) continuing for 2 weeks or more, and detect a mental stress disease at an early stage, and prompt an industrial physician or the like to perform an early intervention.

### Modification of Third Embodiment

### (First Modification)

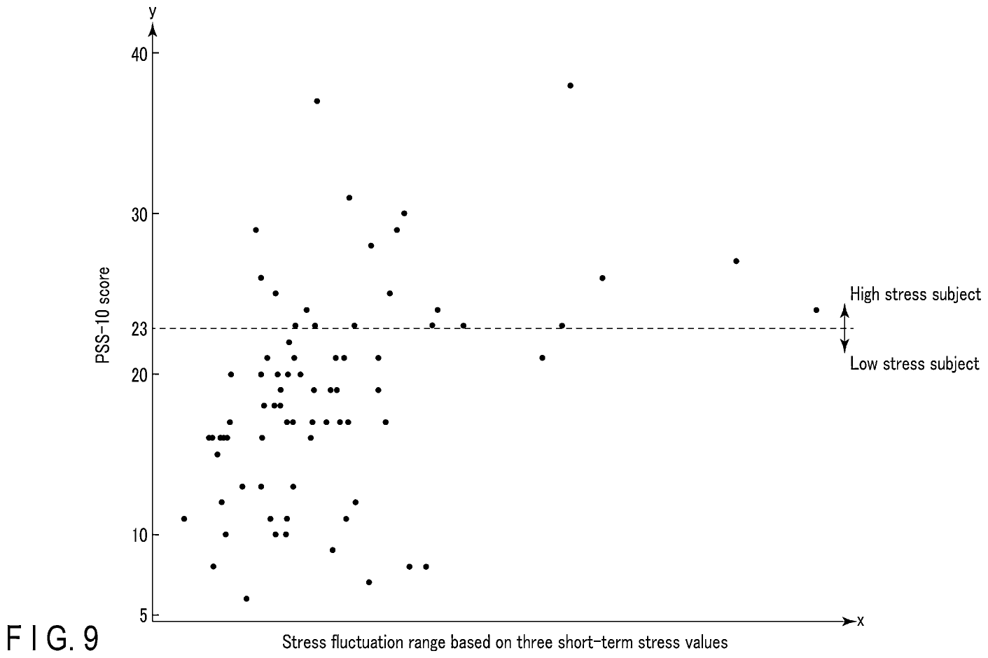

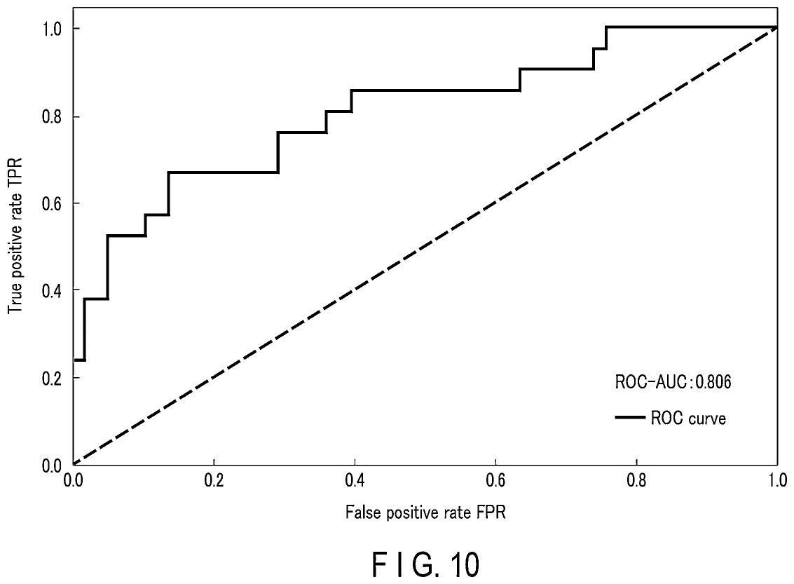

In the third embodiment, a regression model which represents the relationship between the stress fluctuation range based on one kind of short-term stress value and the magnitude of long-term stress is used, but the present invention is not limited to this regression model. For example, in a first modification of the third embodiment, a regression model representing the relationship between the stress fluctuation range based on three types of short-term stress values and the magnitude of long-term stress is used. Here, the stress fluctuation width based on the three types of short-term stress values may be generated, for example, by acquiring three types of biometric waveforms from each of an unspecified multiplicity of subjects to calculate three types of short-term stress values, calculating three types of stress fluctuation widths by removing the baseline from each of the three types of short-term stress values, and combining the three types of stress fluctuation widths. The stress fluctuation range based on the three types of short-term stress values corresponds to the magnitude of long-term stress (the PSS-10 score), as illustrated in an example in FIG. 9. In FIG. 9, the vertical axis represents the PSS-10 score, and the horizontal axis represents the stress fluctuation range based on three types of short-term stress values. There is no multicollinearity between the three types of short-term stress values. Each point shown in FIG. 9 corresponds to each of the 80 unspecified multiplicity of subjects. In FIG. 9, the relationship between the stress fluctuation range on the horizontal axis and the PSS-10 score on the vertical axis has a positive correlation with the (Spearman's rank) correlation coefficient of 0.453 with the multiple regression curve which employs three types of stress fluctuation ranges. In addition, in FIG. 9, a PSS-10 score of 23 points or more is defined as a high stress person, and a PSS score of less than 23 points is defined as a healthy person. Here, the result of the binary classification in a case where the 23 points of the score are set as the threshold value is expressed as an ROC curve indicating the relationship between the false positive rate FPR (False Positive Rate) and the true positive rate TPR (True Positive Rate) as shown in an example in FIG. 10. In FIG. 10, the horizontal axis represents the false positive rate FPR, and the vertical axis represents the true positive rate TPR. The false positive rate FPR and the true positive rate TPR are obtained as shown in the following formulas.

TPR=number of people correctly predicted as having high stress/number of people having high stress

FPR=number of people having low stress erroneously predicted as people having high stress/number of people having low stress

ROC is an abbreviation for Receiver Operating Characteristic. For an ROC curve, it is better to have a high true positive rate TPR and a low false positive rate FPR, and thus a wide surface area under the ROC curve (ROC-AUC) is favorable. In FIG. 10, ROC-AUC is an estimation accuracy of 0.806. In FIG. 10, the oblique broken line represents a case where the ROC-AUC is 0.5.

According to the first modification of the third embodiment as described above, in addition to the above-described advantageous effects, by increasing the types of the stress fluctuation ranges used to estimate the magnitude of long-term stress, an improvement in the estimation accuracy can be expected. Furthermore, in the first modification, stress fluctuation ranges based on three types of short-term stress values are used in the regression model. However, the stress fluctuation ranges based on any plurality of types of short-term stress values can be used in the regression model without being limited to the three types. However, there is no multicollinearity between the plurality of types of short-term stress values.

### Second Modification

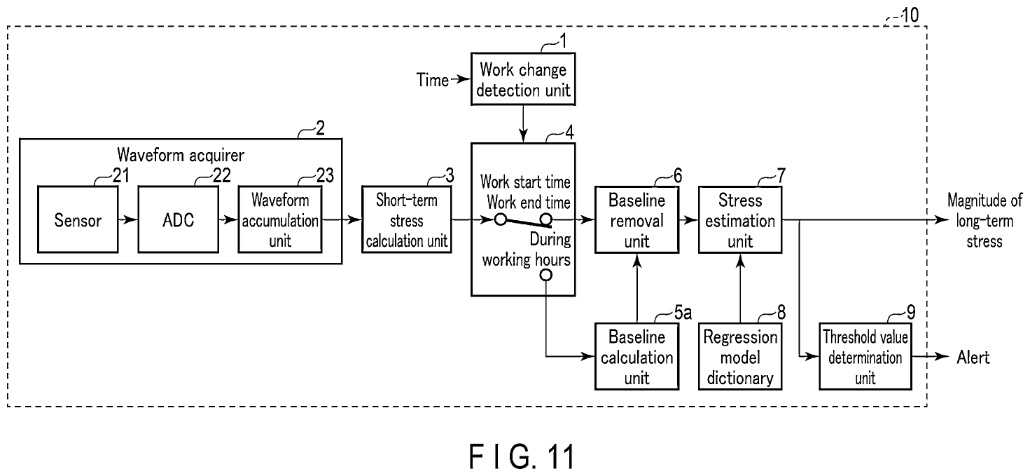

In the third embodiment, the work start detection unit 1a detects the start of work, and the work timing determination unit 4 sends the work-start-time short-term stress values to the baseline removal unit 6, but the present invention is not limited to this arrangement. For example, as illustrated in FIG. 11, the work change detection unit 1 described above may be provided instead of the work start detection unit 1a. Accordingly, based on the result detected by the work change detection unit 1, the work timing determination unit 4 sends the short-term stress value calculated by the short-term stress calculation unit to at least one of the baseline calculation unit 5a and the baseline removal unit 6. For example, the work timing determination unit 4 sends the work-start-time short-term stress values to the baseline removal unit 6 based on the result of detecting the work start time. Furthermore, the work timing determination unit 4 determines the working hours start timing according to whether or not a certain period of time has elapsed since the timing at which the start of work is detected, and thus sends a working-hours short-term stress value to the baseline calculation unit 5a. In addition, the work timing determination unit 4 sends the work-end-time short-term stress values to the baseline removal unit 6 based on the result of detecting the end of work.

The baseline removal unit 6 calculates the user stress fluctuation range by removing the baseline from the sent short-term stress values. For example, the baseline removal unit 6 calculates the stress fluctuation range of the user at the end of work by removing the baseline from the work-end-time short-term stress value thus sent.

Other configurations are the same as those of the third embodiment.

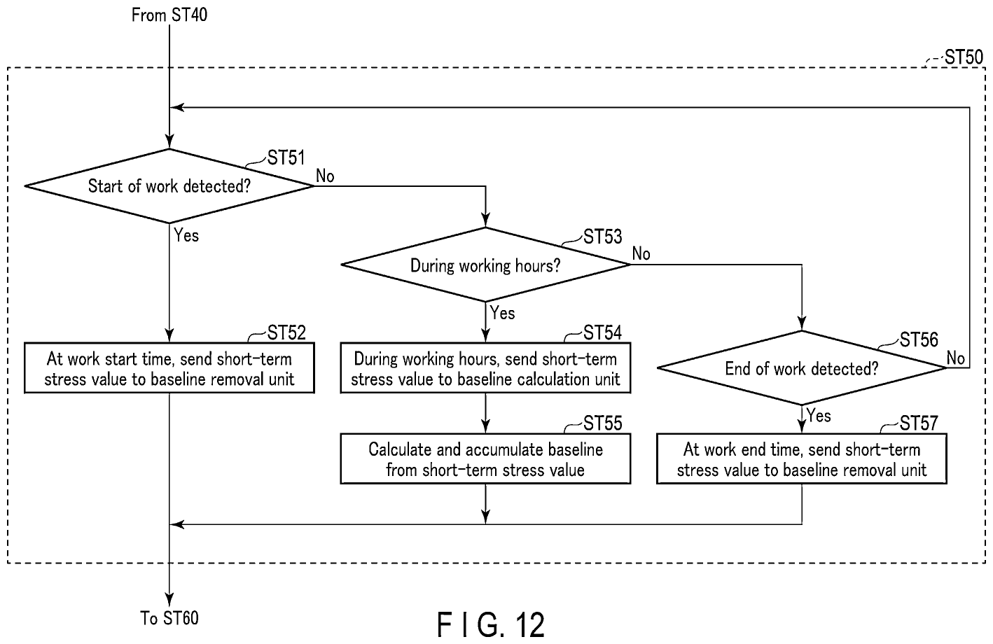

Next, the operation of the stress estimation apparatus configured as described above will be described with reference to the flowchart of FIG. 12. Note that, in the following description, “in the same manner as described above”, the “work start detection unit 1a” in the description using FIG. 5 described above is replaced, as appropriate, with the “work change detection unit 1”, and thus redundant descriptions will be omitted. In addition, in FIG. 12, steps ST56 and ST57 on the right side are added in the middle of returning from step ST53 to step ST51 during step ST50 illustrated in FIG. 5.

That is, it is assumed that steps ST20 to ST51 are executed in the same manner as described above. In a case where the work start time is detected as a result of the determination in step ST51, the processing advances to step ST60 via step ST52 as described above.

In addition, in a case where the determination result of step ST51 is negative, the determination in step ST53 is performed in the same manner as described above, and in a case where the result of the determination is the working hours start timing, the processing advances to step ST60 via steps ST54 and ST55.

On the other hand, in a case where the determination result of step ST53 is negative, the work timing determination unit 4 determines whether the work change detection unit 1 has detected the end of work (step ST56). In the case of a negative determination result, the processing returns to step ST51. In a case where the work end time is detected as a result of the determination in step ST56, the work timing determination unit 4 sends a work-end-time short-term stress value to the baseline removal unit 6 (step ST57), and the processing advances to step ST60.

Hereinafter, the processing in and after step ST60 is executed in the same manner as described above. In the case of a second modification, the baseline removal unit 6 calculates the stress fluctuation range of the user at the work end time by removing the baseline during working hours from the work-end-time short-term stress values, for example (step ST60).

The stress estimation unit 7 estimates the magnitude of the long-term stress of the user based on the work-end-time stress fluctuation range (step ST70), and outputs the estimation result to a display or the like.

According to the second modification of the third embodiment as described above, in addition to the advantageous effects of the third embodiment, it is possible to appropriately estimate the magnitude of long-term stress for a user who is likely to experience stress at the work end time.

### (Third Modification)

In the third embodiment, it is determined whether or not the magnitude of several days' worth of long-term stress falls within a predetermined range, and chronic stress is estimated when the magnitude falls within the predetermined range, but the present invention is not limited to this arrangement. For example, by utilizing a machine learning model using a neural network such as CNN or LSTM, the chronic stress may be estimated based on the magnitude of several days' worth of long-term stress. Even in a third modification of this kind, the same advantageous effects as those of the third embodiment can be obtained.

### (Fourth Modification)

In the fourth embodiment, it is determined whether or not the estimated magnitude of the long-term stress exceeds the threshold value, and an alert is output in a case where the estimated magnitude exceeds the threshold value, but the present invention is not limited to such an arrangement. For example, the alert may be output by accumulating a plurality of determination results spanning days and observing changes in the determination result. Specifically, for example, the alert may be output in a case where all the determination results on a plurality of days exceed the threshold value. According to a fourth modification of this kind, in addition to the above-described advantageous effects, it is possible to reduce situations where a person having low stress is erroneously predicted as a person having high stress.

### Fourth Embodiment

Next, a stress estimation apparatus according to a fourth embodiment will be described.

The fourth embodiment is a specific example or a modification of the first to third embodiments, and represents a calculation example of a baseline, short-term stress values, and a stress fluctuation range.

Here, the work change detection unit 1 detects the start of work and the end of work.

The waveform acquirer 2 acquires a biometric waveform at the detected work start time and a biometric waveform at the detected work end time. In addition, the waveform acquirer 2 acquires a working-hours short-term stress value when a fixed time period has elapsed since the timing at which the work start time is detected.

The short-term stress calculation unit 3 calculates a short-term stress value at the work start time from a biometric waveform at the work start time, and calculates a short-term stress value at the work end time from a biometric waveform at the work end time. Furthermore, the short-term stress calculation unit 3 calculates a working-hours short-term stress value from a working-hours biometric waveform.

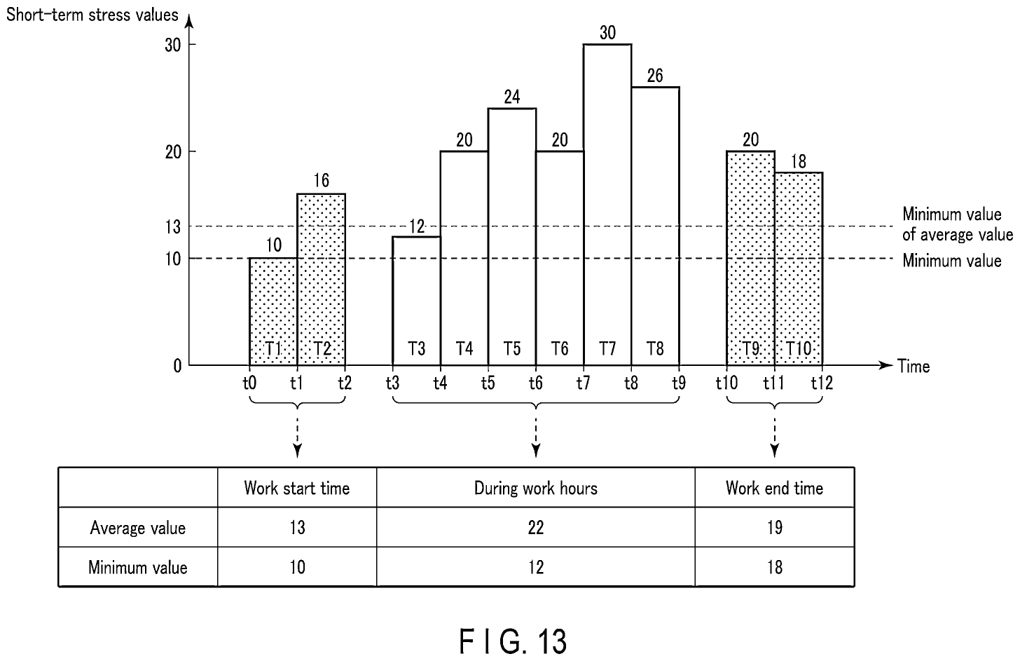

As described above, the baseline accumulation unit 5 and the baseline calculation unit 5a each calculate the baseline based on at least the working-hours short-term stress values. Specifically, when the baseline is to be calculated, as shown in the bar graph of FIG. 13, the work-start-time short-term stress values, the working-hours short-term stress values, and the work-end-time short-term stress values are calculated. In the bar graph of FIG. 13, the vertical axis represents short-term stress values, and the horizontal axis represents time. Note that, in the bar graph, the numerical values of the short-term stress values (10, 16, . . . , 18) are not actual measurement values but, rather, expedient values to facilitate understanding of the calculation method. Similarly, calculated values such as the average values and the smallest values, which are based on the numerical values for the short-term stress values in the bar graph, are also expedient values. In addition, in the bar graph, the short-term stress values are calculated for a fixed period between two times. For example, the short-term stress value (10) is calculated for fixed period T1 between the two times t0 and t1. In the graph, at the start of work, two short-term stress values (10 and 16) are calculated for the two fixed periods T1 and T2. During working hours, six short-term stress values (12, 20, 24, 20, 30, and 26) are calculated for the six fixed periods T3, T4, . . . T8. At the end of work, two short-term stress values (20,19) are calculated for the two fixed periods T9, T10.

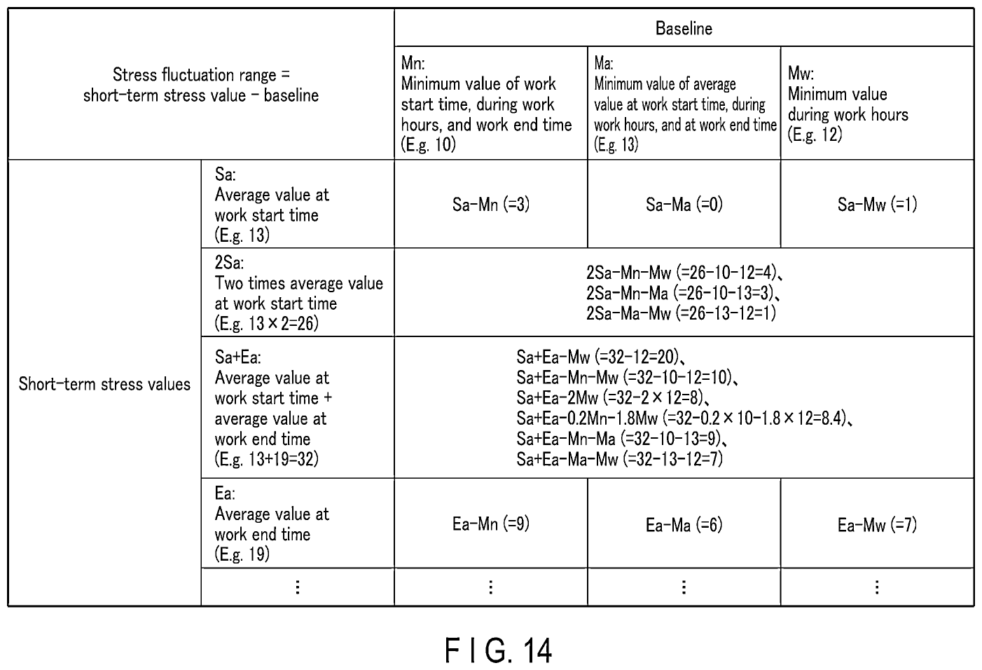

Here, as illustrated in FIGS. 13 and 14, the baseline Ma may be the smallest value (13) among the average value (13) of the work-start-time short-term stress values, the average value (22) of the working-hours short-term stress values, and the average value (19) of the work-end-time short-term stress values.

As illustrated in FIGS. 13 and 14, the baseline Mn may be the smallest value (10) among the work-start-time short-term stress values (10, 16), the working-hours short-term stress value (12, 20, 24, 20, 30, and 26), and the work-end-time short-term stress values (20, 18).

Furthermore, as illustrated in FIG. 14, the baseline Mw may be the smallest value (12) of the working-hours short-term stress values (12, 20, 24, 20, 30, and 26).

Meanwhile, the baseline removal unit 6 calculates the stress fluctuation range by removing the baseline from the average value of the short-term stress values of at least one of the work-start-time short-term stress values and the work-end-time short-term stress values.

For example, as illustrated in FIG. 14, the baseline removal unit 6 may calculate the stress fluctuation range by removing the baseline Mn, Ma, or Mw from an average value Sa of the work-start-time short-term stress values.

In addition, the baseline removal unit 6 may calculate the stress fluctuation range by removing two mutually different baselines among Mn, Ma, and Mw from twice (2Sa) the average value of the work-start-time short-term stress values.

The baseline removal unit 6 may calculate the stress fluctuation range by calculating the total value (Sa+Ea) of the average value Sa of the work-start-time short-term stress values and the average value Ea of the work-end-time short-term stress values and then removing, as the baseline Mw, at least the smallest value among the working-hours short-term stress values from the total value. Note that, in this case, the baseline removal unit 6 may calculate the stress fluctuation range by further removing, as the baseline Mn, the smallest value among the work-start-time short-term stress values, the working-hours short-term stress values, and the work-end-time short-term stress values.

In addition, the baseline removal unit 6 may calculate the stress fluctuation range by removing two baselines among Mn, Ma, and Mw from the total value (Sa+Ea) of the average value Sa of the work-start-time short-term stress values and the average value Ea of the work-end-time short-term stress values. In this case, each of the two baselines to be removed may be multiplied by each of weighting factors (e.g. 0.2, 1.8) that sum to two. In addition, the baseline removal unit 6 may calculate the stress fluctuation range by removing the baseline Mn, Ma, or Mw from the sum (Sa+Ea) of the average value Sa of the work-start-time short-term stress values and the average value Ea of the work-end-time short-term stress values.

Further, the baseline removal unit 6 may calculate the stress fluctuation range by removing the baseline Mn, Ma, or Mw from the average value Ea of the work-end-time short-term stress values.

In addition, the baseline removal unit 6 may calculate the stress fluctuation range by removing two mutually different baselines among Mn, Ma, and Mw from twice (2Ea) the average value of the work-end-time short-term stress values.

As described above, according to the fourth embodiment, the work change detection unit 1 detects the work start time and the work end time. The waveform acquirer 2 acquires a biometric waveform at the detected work start time and a biometric waveform at the detected work end time. The short-term stress calculation unit 3 calculates a short-term stress value at the work start time from a biometric waveform at the work start time, and calculates a short-term stress value at the work end time from a biometric waveform at the work end time. The baseline removal unit 6 calculates the stress fluctuation range by calculating the total value (Sa+Ea) of the average value Sa of the work-start-time short-term stress values and the average value Ea of the work-end-time short-term stress values and then removing, as the baseline Mw, at least the smallest value among the working-hours short-term stress values from the total value. Therefore, in addition to the above-described advantageous effects, the magnitude of long-term stress can be appropriately estimated, based on the short-term stress values at both the work start time and the work end time, for the user who is likely to experience stress at the work start time or the work end time.

Furthermore, according to the fourth embodiment, the baseline removal unit 6 may calculate the stress fluctuation range by further removing, as the baseline Mn, the smallest value among the work-start-time short-term stress values, the working-hours short-term stress value, and the work-end-time short-term stress values. In this case, in addition to the above-described advantageous effects, the magnitude of the calculated stress fluctuation range can be suppressed.

### Fifth Embodiment

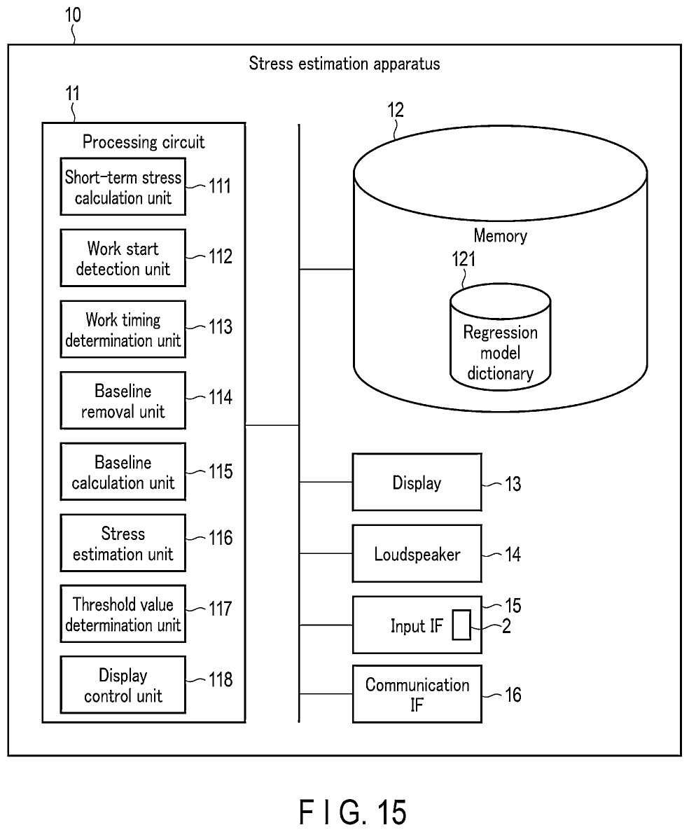

FIG. 15 is a block diagram showing an example of a hardware configuration of the stress estimation apparatus according to the fifth embodiment. The fifth embodiment is a specific example of the first to fourth embodiments, and has a configuration in which the stress estimation apparatus 10 is implemented by a computer. Note that, in FIG. 15, although an Example of units corresponding to the third embodiment is shown, the present invention is not limited to these units, rather, units corresponding to the first, second, and fourth embodiments may be mounted. In the fifth embodiment, the stress estimation apparatus 10 is mounted in the user PC, but the stress estimation apparatus 10 is not limited to such a configuration and may be mounted in a server device capable of communicating with the user PC including the waveform acquirer 2.

The stress estimation apparatus 10 includes, as hardware, a processing circuit 11, a memory 12, a display 13, a loudspeaker 14, an input IF 15, and a communication IF 16. IF signifies interface. Each of the configurations are communicably connected to each other by an internal bus.

The processing circuit 11 controls the entire operation of the stress estimation apparatus 10. The processing circuit 11 includes, as hardware resources, a processor such as a central processing unit (CPU), a micro processing unit (MPU), a graphics processing unit (GPU), a floating point unit (FPU), an application specific integrated circuit (ASIC), and a programmable logic device. For example, the processing circuit 11 executes each program deployed in the memory 12 via the processor to implement the functions of the respective units (e.g. a short-term stress calculation unit 111, a work start detection unit 112, a work timing determination unit 113, a baseline removal unit 114, a baseline calculation unit 115, a stress estimation unit 116, a threshold value determination unit 117, and a display control unit 118) which correspond to each program. Note that the short-term stress calculation unit 111, the work start detection unit 112, the work timing determination unit 113, the baseline removal unit 114, the baseline calculation unit 115, the stress estimation unit 116, and the threshold value determination unit 117 correspond to the short-term stress calculation unit 3, the work start detection unit 1a, the work timing determination unit 4, the baseline removal unit 6, the baseline calculation unit 5a, the stress estimation unit 7, and the threshold value determination unit 9, respectively. Note that each unit can be implemented by a processing circuit 11 which includes a single processor or a processing circuit 11 obtained by combining a plurality of processors.

The memory 12 stores information such as data used by the processing circuit 11, a regression model dictionary 121, and programs. Note that the regression model dictionary 121 corresponds to the regression model dictionary 8 described above. The memory 12 has a semiconductor memory element such as a random access memory (RAM) as hardware. Furthermore, the memory 12 may be a drive device that reads and writes information from and to an external storage device such as a magnetic disk (hard disk), an optical disk (CD-ROM, CD-R, DVD-ROM, DVD-R, or the like), a magneto-optical disk (MO or the like), a semiconductor memory (USB memory, memory card, SSD), or a magnetic tape. The storage area of the memory 12 may be inside the stress estimation apparatus 10 or may be in an external storage device. The memory 12 may accumulate the baseline calculated in advance.

As described above, the program in the memory 12 causes the computer to implement the functions of the respective units. The program includes computer-executable commands, and when executed by the processing circuit 11, causes the processing circuit 11 to execute the series of processes described in relation to the units in FIGS. 1, 4, 6, and 11. For example, when executed by the processing circuit 11, the computer-executable commands included in the program cause the processing circuit 11 to perform a stress estimation method. The stress estimation method may include the steps corresponding to each function of the units described above. In addition, the stress estimation method may, as appropriate, include the steps shown in FIGS. 2, 5, 8, and 12. The memory 12 is an example of a storage unit.

The program may be provided to the stress estimation apparatus 10, which is a computer, in a state of being stored on a computer-readable storage medium. In this case, for example, the stress estimation apparatus 10 further includes a drive (not illustrated) that reads data from the storage medium, and acquires the program from the storage medium. As the storage medium, for example, a magnetic disk, an optical disk, a magneto-optical disk, a semiconductor memory, or the like can be used as appropriate. The storage medium may be referred to as a non-transitory computer-readable storage medium. Alternatively, the program may be stored on a server on the communication network, and the stress estimation apparatus 10 may download the program from the server by using the communication IF 16.

The display 13 is controlled by the display control unit 118 of the processing circuit 11, and displays information such as data generated by the processing circuit 11 and data stored in the memory 12. As the display 13, for example, a display such as a liquid crystal display (LCD), a plasma display, an organic electro-luminescence display (OELD), or a tablet terminal can be used.

The loudspeaker 14 outputs an audio alert based on the alert signal output from the processing circuit 11.

The input IF 15 receives an input from a user using the stress estimation apparatus 10, converts the received input into an electrical signal, and outputs the electrical signal to the processing circuit 11. As the input IF 15, physical operation components such as a mouse, a keyboard, a trackball, a switch, a button, a joystick, a touch pad, a touch panel display, and a microphone can be used. Note that the input IF 15 may be a device that receives an input from an external input device separate from the stress estimation apparatus 10, that converts the received input into an electrical signal, and that outputs the electrical signal to the processing circuit 11. The input IF 15 may include the waveform acquirer 2 in a mouse. In this case, as the waveform acquirer 2, a configuration for acquiring a biometric waveform from a user by means of a mouse-mounted sensor can be used as appropriate. As the mouse-mounted sensor, for example, the above-described photoelectric pulse wave sensor can be used as appropriate. Note that the present invention is not limited to a photoelectric pulse wave sensor, rather, the waveform acquirer 2 may be disposed in a device that can be carried by the user as a component for acquiring a biometric waveform using a biometric sensor, including at least one of an electrocardiographic sensor, a chestband sensor, a wristband sensor, and a smartphone camera (optical camera). The same applies to the following modifications.

The communication IF 16 communicates various kinds of information between the stress estimation apparatus 10 and an external device. Any communication protocol can be used for the communication. Further, the stress estimation apparatus 10 may transmit the estimated magnitude of the long-term stress to the external device via the communication IF 16. The external device may be an operation terminal of an administrator managing the long-term stress of users.

### Modification of Fifth Embodiment

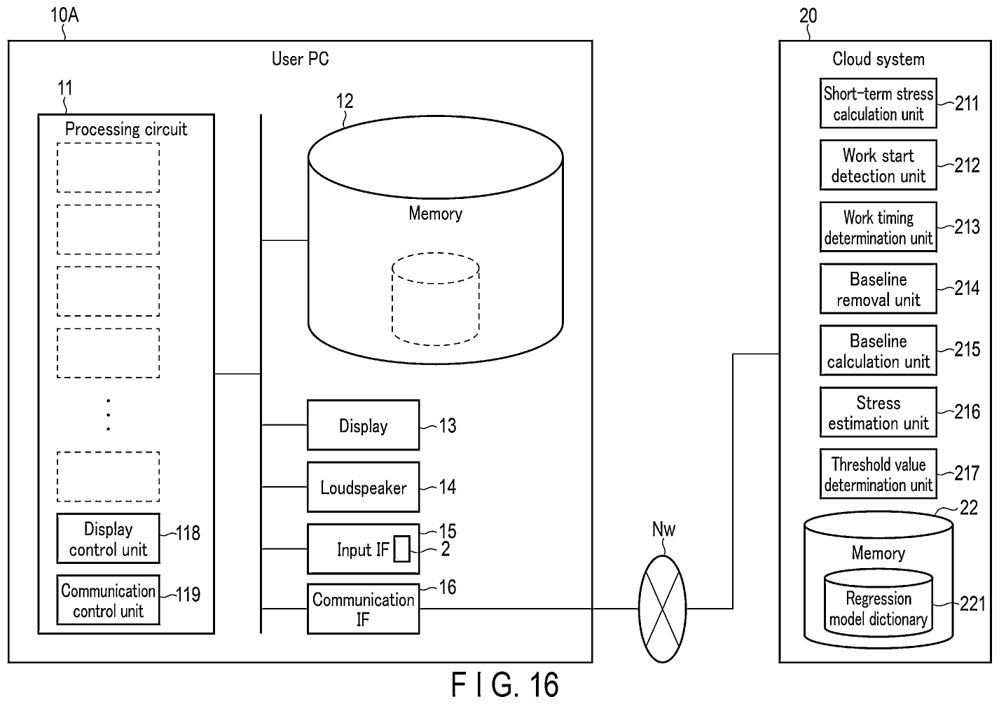

In the fifth embodiment, the functions of the respective units of the stress estimation apparatus 10 are implemented in one computer, but the present invention is not limited to this configuration. For example, as shown in FIG. 16, the functions of the respective units of the stress estimation apparatus 10 may be implemented by the cloud system 20. Accordingly, the stress estimation apparatus 10, which does not have the functions of the respective units, is referred to as a user PC 10A. That is, the stress estimation apparatus according to this modification includes the waveform acquirer 2 mounted in the mouse of the user PC 10A, and includes the cloud system 20 having the functions of the respective units of the above-described stress estimation apparatus 10.

The cloud system 20 is a cloud server in which the functions of a short-term stress calculation unit 211, a work start detection unit 212, a work timing determination unit 213, a baseline removal unit 214, a baseline calculation unit 215, a stress estimation unit 216, and a threshold value determination unit 217 are arranged. Note that the short-term stress calculation unit 211, the work start detection unit 212, the work timing determination unit 213, the baseline removal unit 214, the baseline calculation unit 215, the stress estimation unit 216, and the threshold value determination unit 217 correspond to the short-term stress calculation unit 3, the work start detection unit 1a, the work timing determination unit 4, the baseline removal unit 6, the baseline calculation unit 5a, the stress estimation unit 7, and the threshold value determination unit 9, respectively. The cloud system 20 includes a communication IF and a processing circuit (not illustrated), and a memory 22 including a regression model dictionary 221. In the cloud system 20, the processing circuit executes a program in the memory to implement the functions of the respective units. The cloud system 20 also communicates with the user PC 10A via the communication IF.

Accordingly, the processing circuit 11 of the user PC 10A does not include the above-described units, from the short-term stress calculation unit 111 to the threshold value determination unit 117, and includes the display control unit 118 and the communication control unit 119.

The display control unit 118 fulfils the function of performing display control of the display 13, and for example, causes the display 13 to display the data received from the cloud system 20. As the data, the magnitude of the estimated long-term stress, an alert, or the like, can be used as appropriate, for example.

The communication control unit 119 fulfils the function of controlling the communication with the cloud system 20. For example, the communication control unit 119 transmits the biometric waveform acquired by the waveform acquirer 2 mounted in the mouse to the cloud system 20 via the communication IF16 at the work start time of the user, during working hours, and at the work end time. Furthermore, the communication control unit 119 sends data received from the cloud system 20 via the communication IF to the display control unit 118.

Even in case of the modification as described above, the advantageous effects of the fifth embodiment can be similarly obtained.

According to at least one embodiment described above, it is possible to appropriately estimate the magnitude of long-term stress for the user who is likely to experience stress at the start or end of work. The same applies to at least one modification described above.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A stress estimation apparatus, comprising:
a processing circuit configured to:
detect at least one of a work start time and a work end time for a user performing work during working hours between the work start time and the work end time,
calculate, based on the detected result, a short-term stress value from a biometric waveform of the user acquired by an acquirer,
calculate a baseline based on at least the working-hours short-term stress value,
calculate a stress fluctuation range of the user by removing the baseline from the calculated short-term stress value, and
estimate, based on the stress fluctuation range, a magnitude of long-term stress of the user; and
the acquirer.

2. The stress estimation apparatus according to claim 1,
wherein the processing circuit is configured to detect the work start time,
wherein the acquirer is configured to acquire the biometric waveform at the detected work start time, and
wherein the processing circuit is configured to calculate the stress fluctuation range at the work start time by calculating a short-term stress value at the work start time from the biometric waveform at the work start time and removing the baseline from the short-term stress value at the work start time.

3. The stress estimation apparatus according to claim 1,
wherein the processing circuit is configured to detect the work end time,
wherein the acquirer is configured to acquire the biometric waveform at the detected work end time, and
wherein the processing circuit is configured to calculate the stress fluctuation range at the work end time by calculating a short-term stress value at the work end time from the biometric waveform at the work end time and removing the baseline from the short-term stress value at the work end time.

4. The stress estimation apparatus according to claim 1, wherein the baseline is the smallest value among the work-start-time short-term stress value, the working-hours short-term stress value, and the work-end-time short-term stress value.

5. The stress estimation apparatus according to claim 1, wherein the baseline is the smallest value among an average value of the work-start-time short-term stress values, an average value of the working-hours short-term stress values, and an average value of the work-end-time short-term stress values.

6. The stress estimation apparatus according to claim 1,
wherein the processing circuit is configured to detect the work start time and the work end time,
wherein the acquirer is configured to acquire the detected biometric waveform at the work start time and the detected biometric waveform at the work end time, and
wherein the processing circuit is configured to
calculate a short-term stress value at the work start time from a biometric waveform at the work start time, and calculate a short-term stress value at the work end time from a biometric waveform at the work end time, and
calculate the stress fluctuation range by calculating the total value of the average value of the work-start-time short-term stress values and the average value of the work-end-time short-term stress values and then removing, as the baseline, at least the smallest value among the working-hours short-term stress values from the total value.

7. The stress estimation apparatus according to claim 6, wherein the processing circuit is configured to calculate the stress fluctuation range by also removing, as the baseline, the smallest value among the work-start-time short-term stress values, the working-hours short-term stress values, and the work-end-time short-term stress values.

8. The stress estimation apparatus according to claim 1, wherein the processing circuit is configured to detect at least one of the work start time and the work end time in response to the operations of the user on an office device used for the work.

9. The stress estimation apparatus according to claim 8, wherein the processing circuit is configured to detect the work start time from a startup operation by the user, and detect the work end time from an end operation by the user.

10. The stress estimation apparatus according to claim 9, wherein, in a case where the work start time is to be detected, the processing circuit is configured to detect the work start time in the morning and the work start time in the afternoon separately according to the startup operation and the time.

11. The stress estimation apparatus according to claim 1, wherein, based on the detected result, the processing circuit is further configured to send the calculated short-term stress value to at least one of a calculation processing of the base line and a removal processing of the base line.

12. The stress estimation apparatus according to claim 11, further comprising:
a memory configured to store a regression model representing the relationship between the stress fluctuation range and the magnitude of long-term stress in advance,
wherein the processing circuit is configured to estimate the magnitude of long-term stress of the user based on the regression model.

13. The stress estimation apparatus according to claim 12,
wherein the memory is configured to store the regression model for each type of stress fluctuation width in advance, and
wherein the processing circuit configured to estimate the magnitude of the long-term stress of the user based on the regression model corresponding to the type of the calculated stress fluctuation width.

14. The stress estimation apparatus according to claim 11, wherein the processing circuit configured to output an alert in a case where the estimated magnitude of the long-term stress exceeds a threshold value.

15. The stress estimation apparatus according to claim 11,
wherein the acquirer is configured to acquire the biometric waveform from the user by means of a biometric sensor, and
wherein the biometric sensor includes at least one of an electrocardiographic sensor, a photoelectric pulse wave sensor, a chestband sensor, a wristband sensor, a mouse-mounted sensor, and an optical camera.

16. The stress estimation apparatus according to claim 11,
wherein the acquirer is disposed in the office device used for the work or in a device that can be carried by the user, and
wherein the processing circuit is disposed on a cloud server.

17. A stress estimation method comprising:
detecting at least one of a work start time and a work end time for a user performing work during working hours between the work start time and the work end time;
acquiring a biometric waveform of the user based on the detected result;
calculating a short-term stress value from the biometric waveform;
calculating a baseline based on at least the working-hours short-term stress value;
calculating a stress fluctuation range of the user by removing the baseline from the calculated short-term stress value; and
estimating, based on the stress fluctuation range, the magnitude of long-term stress of the user.

18. A non-transitory computer readable storage medium including computer executable instructions, wherein the instructions, when executed by a processor, cause the processor to perform a method comprising:
detecting at least one of a work start time and a work end time for a user performing work during working hours between the work start time and the work end time;
acquiring, from a sensor, a biometric waveform of the user based on the detected result;
calculating a short-term stress value from the biometric waveform;
calculating a baseline based on at least the working-hours short-term stress value;
calculating a stress fluctuation range of the user by removing the baseline from the calculated short-term stress value; and
estimating, based on the stress fluctuation range, the magnitude of long-term stress of the user.
